(19) United States
(12) Patent Application Publication
(10) Pub. No.: US 2025/0182893 A1
(43) Pub. Date: Jun. 5, 2025

(54) MOTOR FUNCTION IMPROVEMENT ASSISTANCE APPARATUS, MOTOR FUNCTION IMPROVEMENT ASSISTANCE METHOD, AND NON-TRANSITORY COMPUTER-READABLE MEDIUM

(71) Applicants: NEC Corporation, Tokyo (JP); NATIONAL UNIVERSITY CORPORATION TOKYO MEDICAL AND DENTAL UNIVERSITY, Tokyo (JP)

(72) Inventors: Takuya KAWADA, Tokyo (JP); Kazeto YAMAMOTO, Tokyo (JP); Kunihiko SADAMASA, Tokyo (JP); Takuya IBARA, Tokyo (JP); Koji FUJITA, Tokyo (JP); Akimoto NIMURA, Tokyo (JP)

(73) Assignees: NEC Corporation, Tokyo (JP); NATIONAL UNIVERSITY CORPORATION TOKYO MEDICAL AND DENTAL UNIVERSITY, Tokyo (JP)

(21) Appl. No.: 18/845,941

(22) PCT Filed: Feb. 2, 2023
(86) PCT No.: PCT/JP2023/003335

§ 371 Date: Sep. 11, 2024

(30) Foreign Application Priority Data
Mar. 31, 2022 (JP) 2022-058197

(51) Int. Cl. G16H 50/20; G16H 10/60; G16H 20/30

(52) U.S. Cl. CPC G16H 50/20; G16H 10/60; G16H 20/30

(57) ABSTRACT

Provided are a motor function improvement assistance apparatus, a motor function improvement assistance method, and a motor function improvement assistance program that are capable of improving a motor function. The motor function improvement assistance apparatus includes: a background knowledge storage unit that stores background knowledge in which a state of an observable body part and a kinematic problem of the state of the body part are associated with each other, based on a causal relationship; an observation reception unit that receives an observation including examination information and medical interview information of a subject; a hypothesis generation unit that generates, based on the background knowledge and the observation, a plurality of kinematic problem hypotheses by using hypothesis inference; and a hypothesis link generation unit that generates a combination of hypotheses that include an interaction therebetween.

### TECHNICAL FIELD

The present disclosure relates to a motor function improvement assistance apparatus, a motor function improvement assistance method, and a non-transitory computer-readable medium.

### BACKGROUND ART

Patent Literature 1 describes a medical report generation apparatus configured to assist generation of a medical report including a plurality of pieces of information related to a subject of diagnosis and also including a relationship between items in the plurality of pieces of information.

Patent Literature 2 describes an automatic medical interview system configured to receive input data for a medical interview form in which medical interview data are divided into three layers, in order to narrow down symptoms to be determined by the medical interview. The automatic medical interview system reads disease name/clinical department name data associated with the input data, and generates medical interview result data, based on the read disease name/clinical department name data.

Patent Literature 3 describes a rehabilitation assistance apparatus. The rehabilitation assistance apparatus is provided with an elastic ball having a size that can be gripped by a user at a tip thereof, and is configured in such a way that the user performs rehabilitation by handling the elastic ball.

Patent Literature 4 describes a physical function analysis unit configured to acquire physical state information indicating a physical state of a person from one or more sensors for which a person is to be detected, and analyze a change in a physical function of the person, based on a time-series change in the acquired physical state information, and a physical function independence assistance apparatus configured to generate and output physical function improvement proposal information indicating a plan for improving the physical function.

Patent Literature 5 describes a hypothesis verification apparatus configured to derive a hypothesis composed of a logical expression leading to a possible outcome by using knowledge data expressed in the same format from among observation data expressed by a set of logical expressions including one name and one or more parameters.

Patent Literature 6 describes a hypothesis inference apparatus including: a hypothesis generation unit configured to generate a hypothesis candidate set including a hypothesis from which a logical expression is derived, based on knowledge information representing a logical expression deriving a subsequent matter from a previous matter; a conversion unit configured to calculate a constraint condition related to the generated hypothesis candidate set and a weight for the constraint condition; and a solver unit configured to calculate a hypothesis in a case where a predetermined condition is satisfied, based on the calculated constraint condition and the calculated weight.

Patent Literature 7 describes a hypothesis verification apparatus configured to derive a hypothesis candidate having a logical expression leading to a possible outcome by using knowledge data from observation data. The hypothesis verification apparatus adds the logical expression or target logical expression for which authenticity is determined, to the observation data, and derives a hypothesis candidate again.

Patent Literature 8 describes a system configured to assist diagnosis of a patient. The system of Patent Literature 8 provides a primary diagnosis from a combination of a knowledge acquisition module configured to acquire patient data including previous diagnostics, drugs, symptoms, and treatments, and a diagnosis-based predictor, a drug-based predictor, a symptom-based predictor, or a treatment-based predictor.

Patent Literature 9 describes a walking training apparatus including: an actuator configured to assist a trainee's walking motion; a control unit configured to control the actuator according to a setting parameter; and a data acquisition unit configured to acquire a degree of recovery of the trainee.

### CITATION LIST

### Patent Literature

Patent Literature 1: Japanese Unexamined Patent Application Publication No. 2017-033519
Patent Literature 2: International Patent Publication No. WO2011/016447
Patent Literature 3: Japanese Unexamined Patent Application Publication No. 2001-104516
Patent Literature 4: International Patent Publication No. WO2019/187099
Patent Literature 5: International Patent Publication No. WO2019/064600
Patent Literature 6: International Patent Publication No. WO2020/003585
Patent Literature 7: International Patent Publication No. WO2020/170400
Patent Literature 8: Japanese Unexamined Patent Application Publication No. 2017-174407
Patent Literature 9: Japanese Unexamined Patent Application Publication No. 2021-007481

### Non Patent Literature

Non Patent Literature 1: Alberto Garcia Duran, and Mathias Niepert, “Learning graph representations with embedding propagation.” [online], 2017, [Search on Feb. 21, 2022], Internet <https://proceedings.neurips.cc/paper/2017/file/e0688d13958a19e087e123148555 e4b4-Paper.pdf>
Non Patent Literature 2: An open-source cost-based abductive reasoning engine, [online], [Search on Feb. 21, 2022], Internet <https://github.com/naoya-i/henry-n700/>
Non Patent Literature 3: Gurobi Optimization, Inc., Solution Pool, [online], 2016, [Search on Feb. 21, 2022], Internet <http://www.gurobi.com/pdfs/webinars/gurobi-7.0-webinar-slides-de.pdf>
Non Patent Literature 4: Jose A. Gamez, “Abductive Inference in Bayesian Networks: A Review” [online], 2004, [Search on Feb. 21, 2022], Internet <https://link.springer.com/chapter/10.1007/978-3-540-39879-O_6>

### SUMMARY OF INVENTION

### Technical Problem

Patent Literatures 1 to 9 describe an apparatus for diagnosing a disease name and a symptom, but do not describe an apparatus for improving a motor function. An apparatus capable of improving a motor function is desired.

In view of the problem described above, an object of the present disclosure is to provide a motor function improvement assistance apparatus, a motor function improvement assistance method, and a non-transitory computer-readable medium that are capable of improving a motor function.

### Solution to Problem

A motor function improvement assistance apparatus according to an example aspect includes: a background knowledge storage unit configured to store background knowledge in which a state of an observable body part and a kinematic problem of the state are associated with each other based on a causal relationship; an observation reception unit configured to receive an observation including examination information and medical interview information of a subject; a hypothesis generation unit configured to generate, based on the background knowledge and the observation, hypotheses of a plurality of kinematic problems by using hypothesis inference; and an hypothesis link generation unit configured to generate a combination of hypotheses including an interaction between the hypotheses.

A motor function improvement assistance method according to an example aspect includes: storing background knowledge in which a state of an observable body part and a kinematic problem of the state are associated with each other based on a causal relationship; receiving an observation including examination information and medical interview information of a subject; generating, based on the background knowledge and the observation, hypotheses of a plurality of kinematic problems by using hypothesis inference; and generating a combination of hypotheses including an interaction between the hypotheses.

A motor function improvement assistance program according to an example aspect causes a computer to execute: storing background knowledge in which a state of an observable body part and a kinematic problem of the state are associated with each other based on a causal relationship; receiving an observation including examination information and medical interview information of a subject; generating, based on the background knowledge and the observation, hypotheses of a plurality of kinematic problems by using hypothesis inference; and generating a combination of hypotheses including an interaction between the hypotheses.

### Advantageous Effects of Invention

According to the present disclosure, it is possible to provide a motor function improvement assistance apparatus, a motor function improvement assistance method, and a non-transitory computer-readable medium that are capable of improving a motor function.

### BRIEF DESCRIPTION OF DRAWINGS

FIG. 1 is a diagram illustrating an example of a diagnosis made by an automatic diagnosis AI;

FIG. 2 is a diagram illustrating an example of interaction between a plurality of kinematic problems in chronic pain symptoms;

FIG. 3 is a diagram illustrating an example of an input and an output in hypothesis inference according to a first example embodiment;

FIG. 4 is a diagram illustrating an example of a cost and a weight in a weighted abduction according to the first example embodiment;

FIG. 5 is a diagram illustrating an example of a backward reasoning operation in a weighted inference hypothesis according to the first example embodiment;

FIG. 6 is a diagram illustrating an example of a unification operation in the weighted inference hypothesis according to the first example embodiment;

FIG. 7 is a diagram illustrating an example of a configuration that is speeded up by being formulated as an integer linear programming problem in the weighted abduction according to the first example embodiment;

FIG. 8 is a block diagram illustrating an example of a motor function improvement assistance apparatus according to the first example embodiment;

FIG. 9 is a diagram illustrating an example of background knowledge stored in a knowledge DB acquired by a background knowledge storage unit in the motor function improvement assistance apparatus according to the first example embodiment;

FIG. 10 is a diagram illustrating an example of background knowledge stored in the knowledge DB acquired by the background knowledge storage unit in the motor function improvement assistance apparatus according to the first example embodiment;

FIG. 11 is a diagram illustrating an example of background knowledge stored in the background knowledge storage unit at the time of hypothesis estimation in the motor function improvement assistance apparatus according to the first example embodiment;

FIG. 12 is a diagram illustrating an example of a medical interview table accepted by an observation reception unit in the motor function improvement assistance apparatus according to the first example embodiment;

FIG. 13 is a diagram illustrating an example of an observation converted by the observation reception unit at the time of hypothesis estimation in the motor function improvement assistance apparatus according to the first example embodiment;

FIG. 14 is a diagram illustrating an example of a hypothesis generated by a hypothesis generation unit in the motor function improvement assistance apparatus according to the first example embodiment;

FIG. 15 is a diagram illustrating an example of a hypothesis generated by the hypothesis generation unit in the motor function improvement assistance apparatus according to the first example embodiment;

FIG. 16 is a diagram illustrating an example of a hypothesis generated by the hypothesis generation unit in the motor function improvement assistance apparatus according to the first example embodiment;

FIG. 17 is a flowchart illustrating an example of a motor function improvement assistance method according to the first example embodiment;

FIG. 18 is a block diagram illustrating an example of a motor function improvement assistance apparatus according to a second example embodiment;

FIG. 19 is a diagram illustrating an example of integrated plurality of hypotheses in the motor function improvement assistance apparatus according to the second example embodiment;

FIG. 20 is a diagram illustrating an example of a visualized integrated graph structure in the motor function improvement assistance apparatus according to the second example embodiment;

FIG. 21 is a diagram illustrating an example of a visualized integrated graph structure in the motor function improvement assistance apparatus according to the second example embodiment;

FIG. 22 is a diagram illustrating an example of a visualized inference result in the motor function improvement assistance apparatus according to the second example embodiment;

FIG. 23 is a diagram illustrating an example of a visualized inference result in the motor function improvement assistance apparatus according to the second example embodiment;

FIG. 24 is a diagram illustrating an example of a visualized graph structure in the motor function improvement assistance apparatus according to the second example embodiment;

FIG. 25 is a diagram illustrating an example of a visualized graph structure in the motor function improvement assistance apparatus according to the second example embodiment; and

FIG. 26 is a flowchart illustrating an example of a motor function improvement assistance method according to the second example embodiment.

### EXAMPLE EMBODIMENT

Hereinafter, example embodiments is described with reference to the drawings. For clarity of explanation, the following description and the drawings are omitted and simplified as appropriate. In the drawings, the same elements are denoted by the same reference numerals, and redundant descriptions are omitted as necessary.

### First Example Embodiment

A motor function improvement assistance apparatus according to the first example embodiment is described. The motor function improvement assistance apparatus according to the present example embodiment is an apparatus for improving the motor function of a subject person. For example, the motor function improvement assistance apparatus improves the motor function of a person in the field of rehabilitation. In addition, the motor function improvement assistance apparatus improves the motor function of the person in a function training assistance service in which a physical therapist assists individual function training of the person. Furthermore, the motor function improvement assistance apparatus may improve the motor function of a person in the field of training, or may improve the motor function of a person in the field of physical care. Hereinafter, a motor function improvement assistance apparatus used in a rehabilitation field is described as one example. However, the motor function improvement assistance apparatus may be used not only in the field of rehabilitation but also in the field of improving the motor function of a person, such as individual function training, training, and physical care.

In the field of rehabilitation, the subject person is, for example, a patient. In the field of rehabilitation, there is a problem that it is not possible to provide the patient with consistent physiotherapy. This problem includes the following two factors. The first factor is the variability in the quality of treatment and evaluation of physical therapists (hereinafter referred to as “PT”) due to differences in their competence and experience knowledge. The second factor is that the evaluation process is rarely explicitly recorded, making it difficult to accumulate and share experience knowledge. Therefore, if there is a system that can automatically identify the patient's problem from medical interview information and examination information and comprehensively visualize the process thereto, a consistent motor function improvement plan including rehabilitation can be established. At the same time, accumulation and sharing of clinical inference process can be facilitated.

The motor function improvement assistance apparatus of the present example embodiment reproduces the clinical inference process of the PT by applying a hypothesis inference technique, and facilitates assistance of establishing the motor function improvement plan including rehabilitation, and accumulation and sharing of experience knowledge.

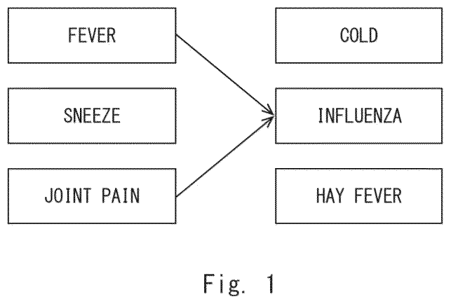

First, before explaining the rehabilitation-specific issues, an automatic diagnosis AI that estimates a pathological condition/disease name from a medical interview/symptoms rather than rehabilitation is explained. This clarifies the rehabilitation-specific issues. FIG. 1 is a diagram exemplifying diagnosis made by the automatic diagnosis AI. As illustrated in FIG. 1, the automatic diagnosis AI estimates a pathological condition/disease name including [cold], [influenza], [hay fever] and the like from a medical interview including [fever], [sneeze], [joint pain] and the like. For example, from a medical interview selecting [fever] and [joint pain], the automatic diagnosis AI diagnoses as [influenza].

As described above, there have been many proposals for an automatic diagnosis AI that predicts and proposes a pathological condition and a disease name from medical interview information input by a patient. The above-mentioned automatic diagnosis AI is sufficient in a case of a symptom in which a countermeasure can be made by predicting the pathological condition from the medical interview information. This is because there is no relationship between the pathological conditions.

However, estimating the pathological condition/disease name from the medical interview/symptom as in the above-mentioned automatic diagnosis AI is insufficient for establishing a motor function improvement plan including rehabilitation and treatment for chronic pain symptoms, represented by non-specific lower back pain, in which the pathological condition/disease name is not clear and for the prevention therefor. In the first place, determining a definite diagnostic name is difficult unless it is a red flag (a symptom that has a necessity of an immediate medical diagnosis such as a malignant tumor). For example, pain in the lower back, shoulder, and neck, are caused by interactions between a plurality of kinematic problems.

Herein, a kinematic problem refers to an abnormality (e.g., excess or insufficiency) of a particular movement (e.g., internal rotation, flexion, etc.) of a particular muscle or joint. Thus, the kinematic problem can be expressed as, for example, “insufficient extension of lumbar spine”.

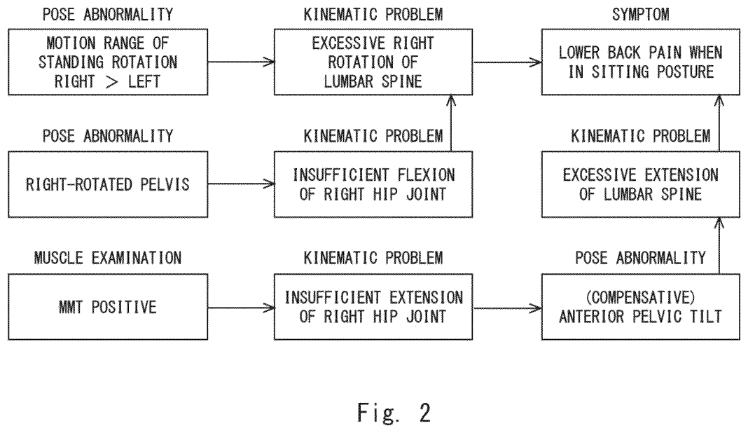

FIG. 2 is a diagram exemplifying the interaction between a plurality of kinematic problems in chronic pain conditions. As illustrated in FIG. 2, from a pose abnormality of “motion range of standing rotation is larger on the right side than on the left side” a kinematic problem of “excessive right rotation of lumbar spine” is estimated. From the kinematic problem of “excessive right rotation of lumbar spine”, a symptom of “lower back pain when in sitting posture” is estimated.

In addition, from a pose abnormality of “right-rotated pelvis”, a kinematic problem of “insufficient flexion of right hip joint” is estimated. From the kinematic problem of “insufficient flexion of right hip joint”, the kinematic problem of “excessive right rotation of lumbar spine” is estimated. From the kinematic problem of “excessive right rotation of lumbar spine”, the symptom of “lower back pain when in sitting position” is estimated.

Furthermore, from a muscle force test result “MMT positive”, a kinematic problem of “insufficient extension of right hip joint” is estimated. From the kinematic problem of “insufficient extension of right hip joint”, a pose abnormality of “(compensative) anterior pelvic tilt” is estimated. From the pose abnormality of “(compensative) anterior pelvic tilt”, a kinematic problem of “excessive extension of lumbar spine” is estimated. From the kinematic problem of “excessive extension of lumbar spine”, the symptom of “lower back pain when in sitting position” is estimated.

As illustrated in FIG. 2, the case of “lower back pain when in sitting position” is caused by the interaction between a plurality of kinematic problems. Therefore, in a case of chronic pain symptoms such as pain in the lower back, shoulder, and neck, the kinematic problem of the patient and its interaction are first clarified from medical interview information and examination information. Then, a rehabilitation plan can be established for the first time after grasping an overall image of the condition of the patient causing the lower back pain or the like. In some cases, there may be a plurality of interactions acquired from the same medical interview information and examination information (a graph in which kinematic problems are connected by an arrow as in FIG. 2), and therefore, a comprehensive estimation of a solution is necessary.

For chronic pain symptoms such as pain in the lower back, shoulder, and neck and prevention therefor, the following matters need to be clarified in order to establish a motor function improvement plan including rehabilitation and treatment.

### Patient's Kinematic Problems

Interaction between each kinematic problem (how one kinematic problem is associated with other kinematic problems, and how such kinematic problems are associated with the pain the patient is suffering from)

Since the kinematic problems and the interactions thereof are not uniquely defined, it is necessary to comprehensively present the possibilities. However, in a case of an inexperienced PT, other possibilities may be overlooked. Therefore, if only one solution is acquired, it is not possible to achieve establishment of a consistent motor function improvement plan.

As a technique for predicting the condition of a patient from observation information such as medical interview information and examination information and predicting the relation as a graph structure, there are graph AI and hypothesis inference. For example, as described in Non-Patent Literature 1, the graph AI can output a graph structure when learning data relative to input and output are prepared. Therefore, even in the task of lower back pain, if a large amount of data relative to input and output can be prepared, it is possible to predict some kinds of kinematic problems and relevant links thereof. One example of training and execution of the graph AI is described below.

At the time of training, a link between a patient and his/her attributes as described below is prepared and learned.

[Patient id: 001]-age-[40], [Patient id: 001]-body temperature-[38.2], [Patient id: 001]-has-[cough], [Patient id: 001]-has-[influenza]

[Patient id: 002]-age-[16], . . .

At the time of execution, it is predicted whether a link is established, based on data in which a part of a link between nodes is unknown.

(Example of input) [Patient id: 100]-age-[36], [Patient id: 001]-body temperature-[36.2], [Patient id: 100]-???-[influenza]

(Output example) [Patient id: 100]-has-[influenza]

However, there is a problem in applying the graph AI to the field of rehabilitation. In the field of rehabilitation, it is necessary to satisfy the following requirements in order to provide consistent physiotherapy for patients. The first requirement is the identification of the plurality of kinematic problems that the patient suffers. The second requirement is the identification of the interaction of each kinematic problem. In addition, since there are constraints (exclusive relationships) in the relationships between kinematic problems, the estimation of constrained interactions following kinematics is required. However, the graph AI can learn link relationships between nodes, but cannot learn (at least cannot control) exclusive relationships between nodes. Therefore, it is impossible to estimate the constrained interactions.

For example, since “insufficient movement in extension direction of lumbar spine” and “excessive movement in extension direction of lumbar spine” do not co-occur, these matters are in an exclusive relationship. Also, since “lumbar lordosis increase” and “lumbar lordosis decrease” do not co-occur, these matters are in an exclusive relationship. Comprehensive presentation of constrained interactions requires to derive a plurality of possible solutions. However, the graph AI can present only one solution.

Therefore, the present example embodiment provides a motor function improvement assistance apparatus to which hypothesis inference is applied, that enables estimation of constrained interactions following kinematics and that can present multiple solutions. Hereinafter, <hypothesis inference> and <weighted abduction> used by the motor function improvement assistance apparatus according to the present example embodiment is described.

### <Hypothesis Inference>

Hypothesis inference is also called idea inference or abduction. The hypothesis inference derives reasonable hypothesis from background knowledge (also called rules or inference knowledge) given by a logical expression and an observed event (also called acquired fact. Note that, in the following description, the observed event is simply referred to as an observation). For example, suppose that there is a rule “B holds if A holds” (A implies B), and that “B holds” is observed. The hypothesis inference is an inference method that, at this time, assumes that “B is true because A is true” and thereby makes a hypothesis that “A is true”. It is also called “backward reasoning” due to viewing the rules backward.

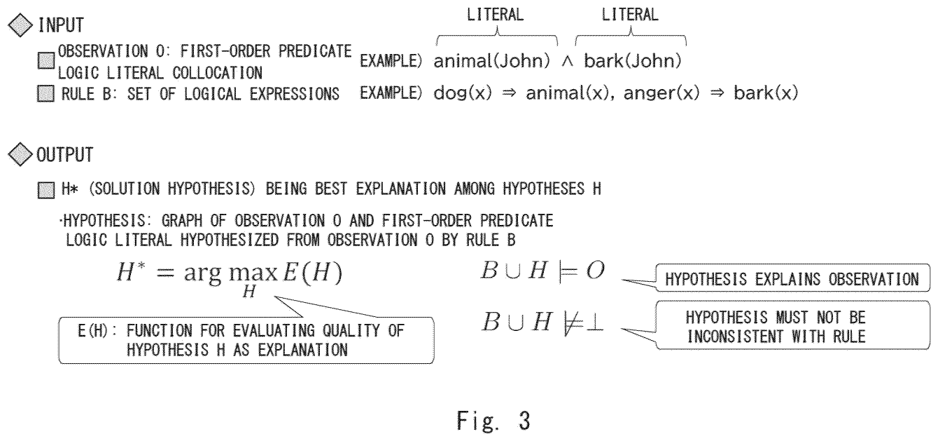

FIG. 3 is a diagram exemplifying an input and an output in the hypothesis inference according to the first example embodiment. As illustrated in FIG. 3, in the hypothesis inference, the inputs are observation O and rule B. The observation O includes a collocation of first-order predicate logic literals. For example, expression (1) described below is used. Note that the expression is also illustrated in FIG. 3 in order to describe the exact symbols of the following expressions.

$$animal ( John ) ⋀ bark ( John ) \qquad ( 1 )$$

Herein, animal(John) is a literal and bark(John) is a literal. A literal is an elementary logical expression p(t1, t2, . . . ) or an elementary logical expression with a negative sign (¬ or !). In a case where a term, that is, a literal argument (t1, t2, . . . ), is a character string that starts in uppercase letters or is enclosed in quotation marks, the term indicates a constant, otherwise, the term is a variable.

Rule B is a set of logical expressions. For example, the rule B is a set of logical expressions as represented by expressions (2) and (3).

$$dog ( x ) ⇒ animal ( x ) \qquad ( 2 )$$

$$anger ( x ) ⇒ bark ( x ) \qquad ( 3 )$$

In hypothesis inference, the output is H* (solution hypothesis) which is the best explanation among the hypotheses H. Herein, the hypothesis includes a graph of the observation O and the first-order predicate logical literal hypothesized by the rule B from the observation O. For example, equation (4) described below is used.

$$H *= arg {max}_{H} E ( H ) \qquad ( 4 )$$

Herein, E(H) is an evaluation function that evaluates the goodness of the hypothesis H as an explanation.

In general, evaluation functions evaluate the hypothetical goodness of candidates for hypotheses generated from observations and background knowledge in hypothesis inferences. The evaluation function may output the goodness of the generated hypothesis candidate as a numerical value (score). The hypothesis inference based on the evaluation function outputs the hypothesis by evaluation of the evaluation function from among candidates of the hypothesis generated from the observation and the background knowledge.

Equation (5) described below indicates that the hypothesis describes the observation.

$$B ⋃ H \vert = O \qquad ( 5 )$$

Inequality (6) described below indicates that the hypothesis should not be inconsistent with the rule.

$$B ⋃ H \vert = ⊥ \qquad ( 6 )$$

### <Weighted Abduction>

Next, weighted abduction is explained. Weighted abduction is one of the hypothesis inference methods. Weighted abduction generates hypothesis candidates by applying backward reasoning operations and unification operations. In the weighted abduction, as in equation (7) described below, a hypothesis candidate having a smaller sum of the total costs is a better explanation. In the weighted inference, the score of the candidate hypothesis is calculated by the cost using the evaluation function of equation (7).

$$E ( H ) = - {\sum }_{p ∈ H} cost ( p ) \qquad ( 7 )$$

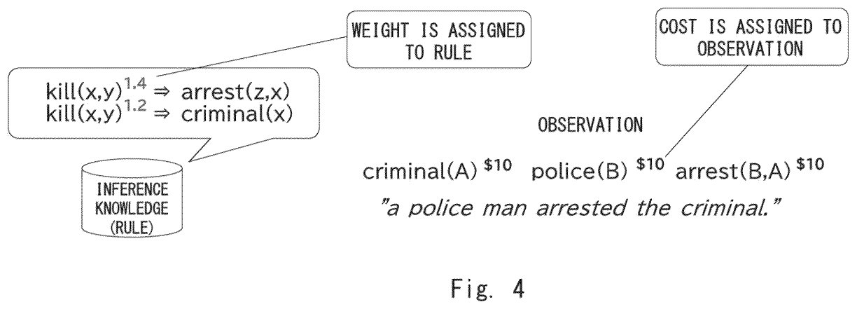

FIG. 4 is a diagram illustrating costs and weights in the weighted abduction according to the first example embodiment. As illustrated in FIG. 4, for example, observations of the following expressions (8) to (10) acquired from “a police man arrested the criminal (police officer (B) arrested criminal (A))” are each assigned a cost of $10. Expression (8) indicates that A is a criminal, expression (9) indicates that B is a police officer, and expression (10) indicates that B has arrested A. The “cost” assigned to (the literal of) the observation indicates how much the literal should be explained.

$$criminal {( A )}^{\$10} \qquad ( 8 )$$

$$police {( B )}^{\$10} \qquad ( 9 )$$

$$arrest {( B , A )}^{\$10} \qquad ( 10 )$$

Meanwhile, the “weight” assigned to the rule indicates how unreliable it is to hypothesize the previous matter from the subsequent matter. For example, the background knowledge (rules) of the following expressions (11) and (12) are assigned weights of, for example, 1.4 and 1.2.

$$kill {( x , y )}^{1.4} ⇒ arrest ( z , x ) \qquad ( 11 )$$

$$kill {( x , y )}^{1.2} ⇒ criminal ( z , x ) \qquad ( 12 )$$

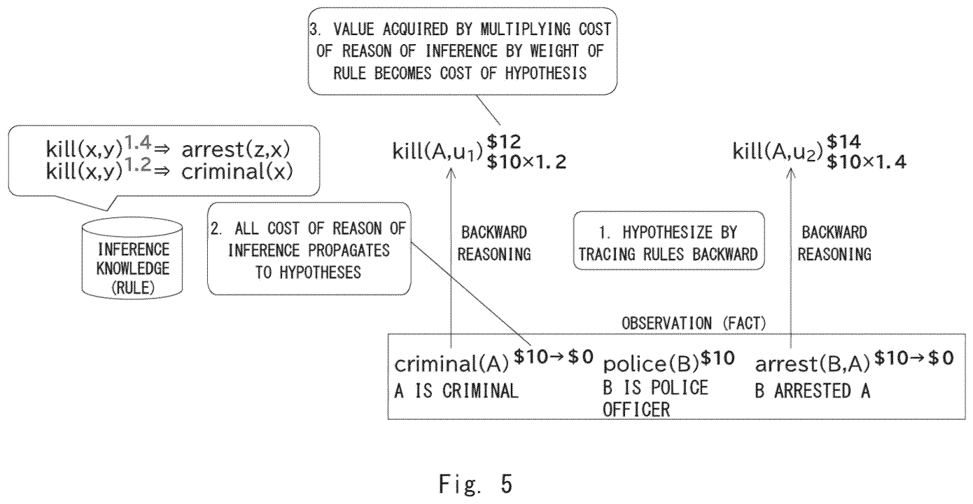

Next, a backward reasoning operation and a unification operation applied to a weighted inference hypothesis is explained. FIG. 5 is a diagram exemplifying a backward inference operation in the weighted inference hypothesis according to the first example embodiment. As illustrated in FIG. 5, the backward inference operation includes operations 1 to 3.

First, in the backward reasoning, as operation 1, a hypothesis is made by tracing the rule backward. For example, “kill(x, y)^{1.2}→criminal(z, x)” in expression (12) is traced backward based on “criminal(A)^{$10}” in expression (8). Then, expression (13) is acquired.

$$kill {( A , {u}_{1} )}_{\$10 \times 1.2}^{\$12} \qquad ( 13 )$$

Further, for example, “kill(x, y)^{1.4}→arrest(z, x)” in expression (11) is traced backward based on “arrest(B, A)^{$10}” in expression (10). Then, expression (14) is acquired.

$$kill {( A , {u}_{2} )}_{\$10 \times 1.4}^{\$14} \qquad ( 14 )$$

Herein, in the backward reasoning, as operation 2, all the costs of the reason for inference is propagated to the hypothesis. Therefore, as indicated by expression (15) described below, the “criminal(A)^{$10}” of expression (8) becomes $0. As indicated by expression (16) described below, the “arrest(B,A)^{$10}” in expression (10) becomes $0.

$$criminal {( A )}^{\$10 \to \$0} \qquad ( 15 )$$

$$arrest {( B , A )}^{\$10 \to \$0} \qquad ( 16 )$$

Meanwhile, in the backward reasoning, as operation 3, a value acquired by multiplying the cost of the reason of the inference by the weight of the rule is the cost of the hypothesis. Therefore, as described in expression (13), the cost of expression (12) is $12. As described in expression (14), the cost of expression (11) is $14.

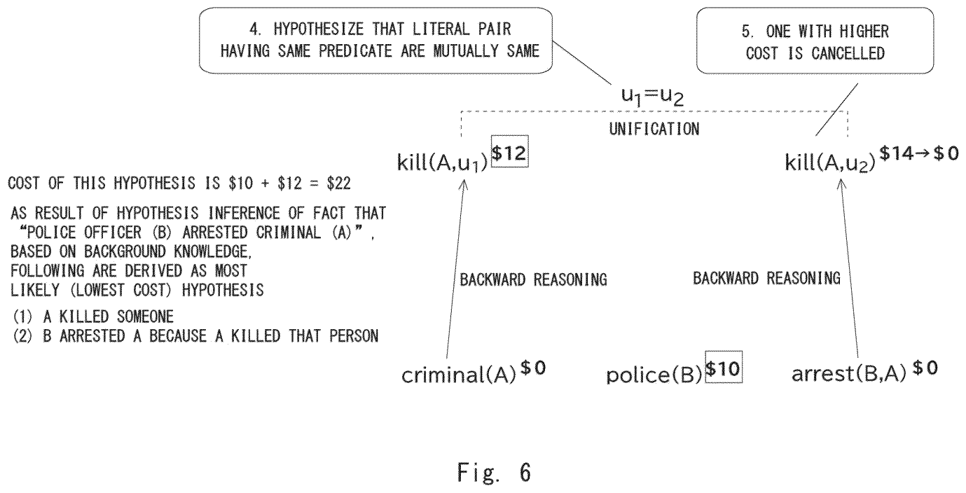

Next, the unification operation is explained. FIG. 6 is a diagram exemplifying a unification operation in the weighted inference hypothesis according to the first example embodiment. As illustrated in FIG. 6, in the unification, as operation 4, a hypothesis is made that literal pairs having the same predicate are identical to each other. For example, expressions (13) and (14) described above have the same predicate. Therefore, the literal pairs are the same as each other. Thus, the following equation (17) is acquired.

$${u}_{1} = {u}_{2} \qquad ( 17 )$$

Next, in the unification, as operation 5, the higher one of the costs is cancelled. Comparing expression (13) with expression (14), the cost in expression (14) is higher. Thus, the cost of expression (14) is cancelled, resulting in expression (18).

$$kill {( A , {u}_{2} )}^{\$14 \to \$0} \qquad ( 18 )$$

Eventually, expressions (8) to (10) become the following expressions (19) to (21), and expressions (11) and (12) become expressions (22) and (23).

$$criminal {( A )}^{\$0} \qquad ( 19 )$$

$$police {( B )}^{\$10} \qquad ( 20 )$$

$$arrest {( B , A )}^{\$0} \qquad ( 21 )$$

$$kill {( A , {u}_{1} )}^{\$12} \qquad ( 22 )$$

$$kill {( A , {u}_{2} )}^{\$0} \qquad ( 23 )$$

The cost of this hypothesis is $10+$12=$22. Therefore, as a result of the hypothesis inference of the fact that “police officer (B) arrested the criminal (A)”, based on the background knowledge, the following is derived as a likely (lowest cost) hypothesis.
(1) A killed a person.
(2) B arrested A because A killed the person.

### <Acceleration of Weighted Abduction by ILP/SAT>

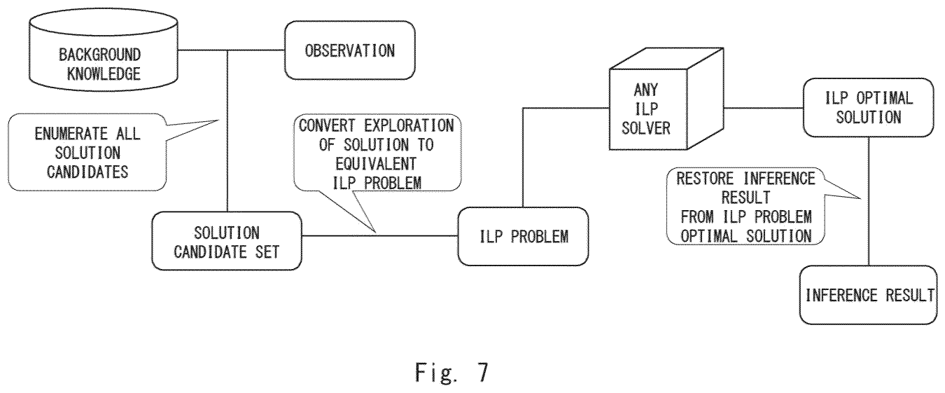

The search for solutions in hypothesis inference can be accelerated by formulating as an integer linear programming problem (ILP) or a satisfiability problem (SAT). FIG. 7 is a diagram exemplifying a configuration, in the weighted abduction according to the first example embodiment, that is formulated as an integer linear programming problem and thus accelerated. As illustrated in FIG. 7, based on background knowledge and observations, all candidate solutions in the weighted abduction are enumerated to generate a set of candidates. Then, the search for the solution is converted into an equivalent ILP problem. Next, an optimal solution of the ILP problem is derived using an arbitrary ILP solver. Then, the inference result is restored from the optimal solution of the ILP problem.

As the ILP solver, for example, one disclosed as open source software in Non Patent Literature 2 may be used. For example, the SAT solver described in Patent Literature 6 may be used to increase the speed. Either of them is applicable not only to weighted abduction but also to hypothesis inference based on an evaluation function.

<k-Best Solution of Weighted Abduction>

Next, a k-best solution of the weighted abduction is explained. The weighted abduction can output of the k-best solution by using a solver that can output multiple solutions among solvers being used by an inference engine such as an ILP solver. Herein, the k-best solution is a plurality of solutions the evaluation function values of which are equivalent.

A solver in which a multiple solution outputting functions are implemented is one described in, for example, Solution Pool (page 18) of Non Patent Literature 3. As described in Non Patent Literature 4, the output method of the k-best solution in the hypothesis inference is also known. However, none of them are intended for hypothesis verification models. A method for integrating the k-best solutions is described in, for example, Patent Literature 7.

### <Motor Function Improvement Assistance Apparatus>

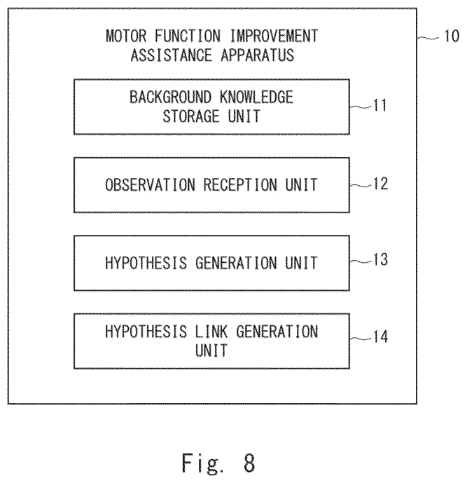

Next, a motor function improvement assistance apparatus according to the present example embodiment is explained. FIG. 8 is a block diagram exemplifying a motor function improvement assistance apparatus according to the first example embodiment. As illustrated in FIG. 8, a motor function improvement assistance apparatus 10 includes a background knowledge storage unit 11, an observation reception unit 12, a hypothesis generation unit 13, and a hypothesis link generation unit 14. The background knowledge storage unit 11, the observation reception unit 12, the hypothesis generation unit 13, and the hypothesis link generation unit 14 function as a background knowledge storage means, an observation reception means, a hypothesis generation means, and a hypothesis link generation means.

The background knowledge storage unit 11 stores background knowledge (rules). In the background knowledge, for example, a state of an observable body part is associated with a kinematic problem of the state of the body part, based on a causal relationship. Specifically, background knowledge includes causal relationships and constraints of observable body part information, such as pain and pose abnormalities, and relative kinematic issues. The state of the observable body part includes lifestyle habits and the state of the body part, such as movement of joints and muscles. The background knowledge storage unit 11 may store, as background knowledge, exclusive relationships of a plurality of kinematic problems that cannot be simultaneously established. The background knowledge storage unit 11 stores background knowledge of the following examples 1 to 3, for example. However, the background knowledge is not limited to examples 1 to 3.

Example 1: Insufficient flexion of right hip joint implies Buttocks pain during lumbar extension.

Example 2: Lumbar spine lordosis decreased implies Excessive extension of cervical spine.

Example 3: Lumbar spine lordosis decrease and lumbar spine lordosis increase are in an exclusive relationship (not concomitant).

The background knowledge storage unit 11 may store background knowledge in a knowledge database (referred to as a knowledge DB). Then, the background knowledge storage unit 11 may convert the background knowledge in the knowledge DB into a logical expression and use the logical expression at the time of hypothesis reasoning.

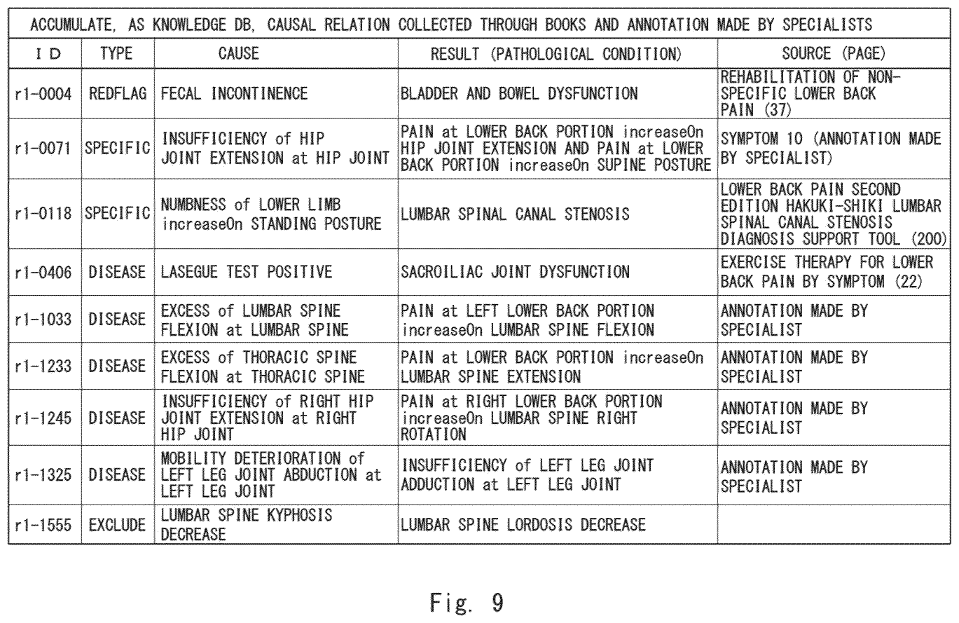

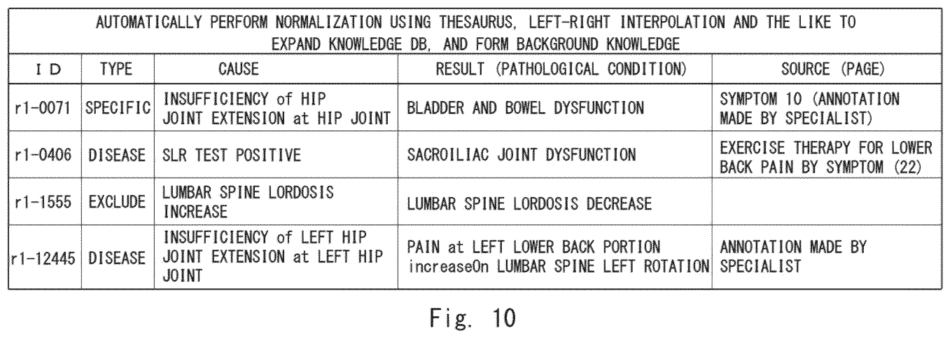

FIGS. 9 and 10 are diagrams exemplifying the motor function improvement assistance apparatus 10 according to the first example embodiment, wherein background knowledge stored in the knowledge DB acquired by the background knowledge storage unit 11 is illustrated. As illustrated in FIG. 9, the background knowledge storage unit 11 may accumulate the causal relationship collected from a book or an annotation of a specialist as the knowledge DB. Further, as illustrated in FIG. 10, the background knowledge storage unit 11 may automatically perform normalization using a synonym dictionary, left and right complementation, or the like, and extend the knowledge DB and convert the knowledge DB into background knowledge.

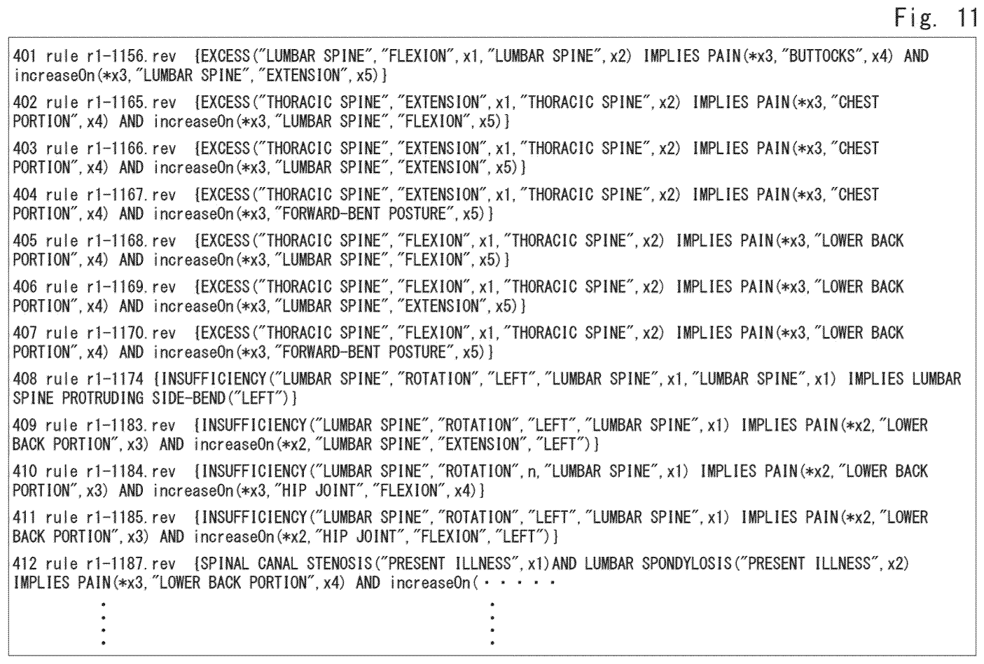

FIG. 11 is a diagram exemplifying background knowledge stored in the background knowledge storage unit 11 at the time of hypothesis estimation in the motor function improvement assistance apparatus 10 according to the first example embodiment. As illustrated in FIG. 11, the background knowledge storage unit 11 converts the background knowledge stored in the knowledge DB into a predetermined format that can be read by the information processing apparatus and stores the converted background knowledge.

The observation reception unit 12 receives an observation. The observation includes, for example, medical interview information and examination information of the subject, and the like.

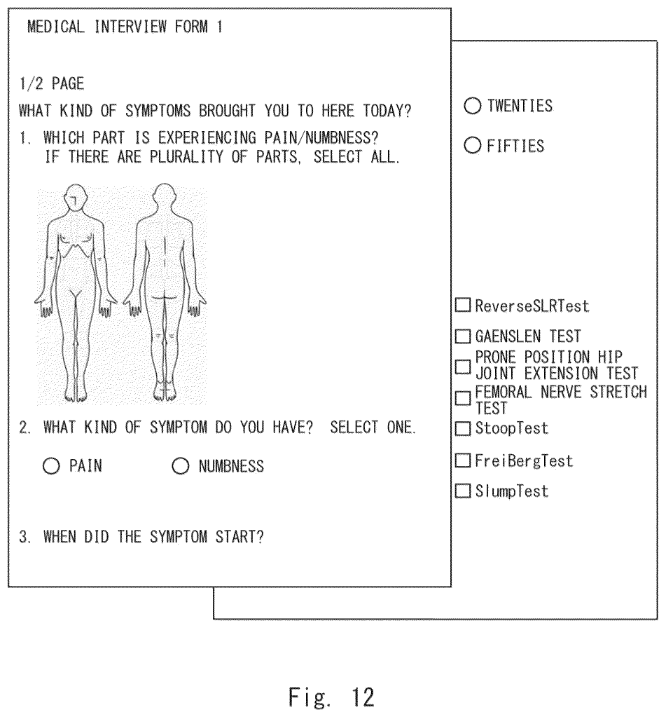

FIG. 12 is a diagram exemplifying a medical interview table received by the observation reception unit 12 in the motor function improvement assistance apparatus 10 according to the first example embodiment. As illustrated in FIG. 12, the observation reception unit 12 may receive the medical interview information and the examination information via the medical interview form UI based on an actual interview form or examination items. Input from a smartphone or a tablet terminal is enabled by using the medical interview form UI. In addition, since the observation reception unit 12 receives the observation of the medical interview information, the examination information, and the like, it is possible to simplify the input of pain location and medical interview examination information.

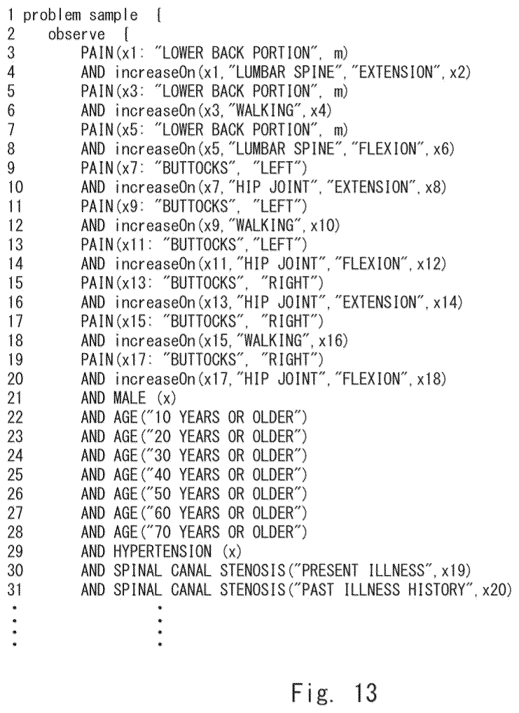

FIG. 13 is a diagram exemplifying an observation converted by the observation reception unit 12 at the time of hypothesis estimation in the motor function improvement assistance apparatus 10 according to the first example embodiment. As illustrated in FIG. 13, the observation reception unit 12 converts the received observation into a predetermined format that can be read by the information processing apparatus.

The hypothesis generation unit 13 generates a hypothesis. For example, the hypothesis generation unit 13 generates a hypothesis of a plurality of kinematic problems using hypothesis inference based on background knowledge and observation. Specifically, the hypothesis generation unit 13 may perform hypothesis inference by combining background knowledge and observation, and enumerate a plurality of kinematic problems. The hypothesis generation unit 13 may generate a hypothesis based on background knowledge including an exclusive relationship. Further, the hypothesis generation unit may evaluate each hypothesis using an evaluation function that evaluates the generated hypothesis.

For example, the hypothesis generation unit 13 may generate a plurality of hypotheses composed of logical expressions for which truth is to be verified by using a kind of hypothesis inference that can derive multiple solutions, based on an evaluation function while taking into consideration constraints represented by weighted abduction. Specifically, the hypothesis generation unit 13 selects a plurality of generated hypothesis candidates in consideration of constraints such as an exclusive relationship. Then, the hypothesis generation unit 13 evaluates the selected hypothesis candidates by an evaluation function, and further selects hypothesis candidates having a predetermined value or more. In such a way, the hypothesis generation unit 13 may generate a hypothesis. As one example, the hypothesis generation unit 13 may generate a hypothesis by weighted abduction that applies a backward inference operation and a unification operation. In such a case, the hypothesis generation unit 13 generates a hypothesis from among the candidates of the hypothesis by evaluation based on the cost.

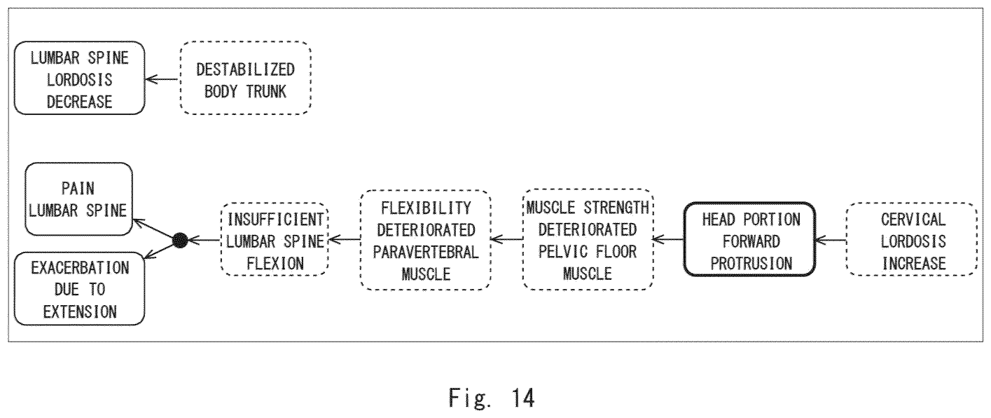

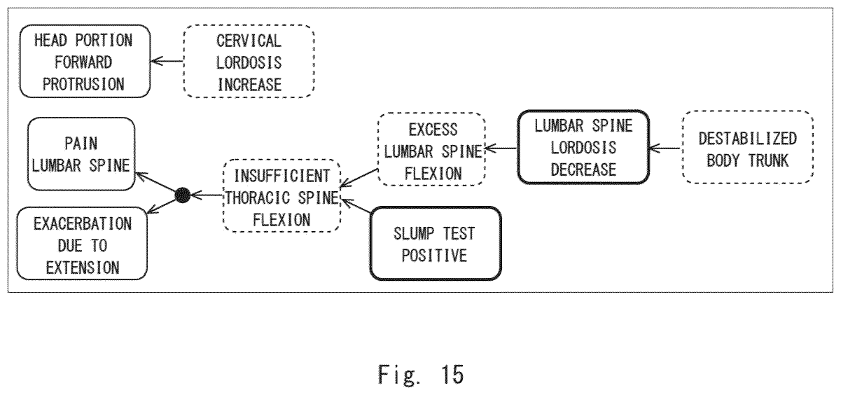

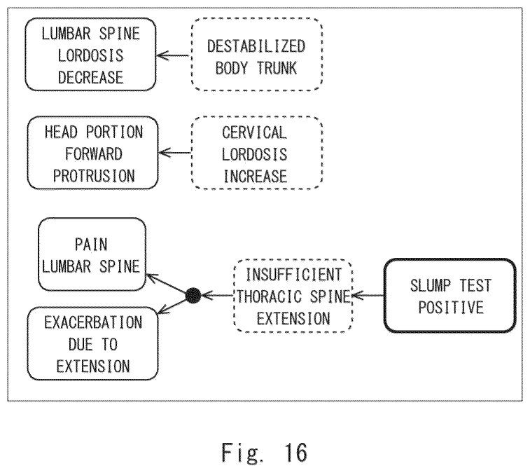

FIGS. 14 to 16 are diagrams exemplifying hypotheses generated by the hypothesis generation unit 13 in the motor function improvement assistance apparatus 10 according to the first example embodiment. As illustrated in FIGS. 14 to 16, items such as “lumbar spine lordosis decrease”, “lumbar spine pain (pain in lumbar spine)”, “exacerbation due to extension”, and “head forward protrusion” surrounded by thin lines and thick lines indicate medical interview information and examination information. Items such as “destabilized body trunk”, “insufficient lumbar spine flexion (insufficiency in flexion of lumbar spine)”, “flexibility deteriorated paravertebral muscle”, “muscle strength deteriorated pelvic floor muscle”, and “cervical lordosis increase” surrounded by dotted lines indicate kinematic problems hypothesized from medical interview information and examination information. The hypothesis generation unit 13 generates hypotheses of a plurality of kinematic problems from the observation of medical interview information, examination information, and the like.

The hypothesis generation unit 13 may generate hypotheses of a plurality of kinematic problems by formulating as an integer linear programming problem or a satisfiability problem described above. Further, the hypothesis generation unit 13 may generate a plurality of hypotheses having the same evaluation function value for evaluating the plurality of generated hypotheses by using a solver capable of outputting multiple solutions.

The hypothesis link generation unit 14 generates a combination of hypotheses including an interaction between the hypotheses. Specifically, the hypothesis link generation unit 14 may generate the interaction between the hypothesized kinematic problems as a graph structure. FIGS. 14 to 16 are diagrams illustrating a plurality of hypotheses each having the same evaluation function value as a graph structure. Interactions among the hypotheses of the kinematic problems illustrated in each graph (the items surrounded by dotted lines) are represented by connections with arrows. The hypothesis link generation unit 14 may output, as a graph structure, kinematic problems that can best explain information that can be observed based on the background knowledge, and the relationship therebetween. The hypothesis link generation unit 14 may generate an appropriate combination of kinematic problems and a reason thereof, based on the evaluation function.

For example, as illustrated in FIG. 14, the hypothesis link generation unit 14 indicates, by the connections with arrows, that there is an interaction between “insufficient lumbar spine flexion (insufficiency in flexion of lumbar spine)” and “flexibility deteriorated paravertebral muscle” surrounded by dotted lines and that there is an interaction between “flexibility deteriorated paravertebral muscle” and “muscle strength deteriorated pelvic floor muscle”. This makes it possible to comprehensively reproduce the inference process from the medical interview information and the examination information of “head portion forward protrusion” to “lumbar spine pain (pain in lumbar spine)” and “exacerbation due to extension” via the kinematic problems of “muscle strength deteriorated pelvic floor muscle”, “flexibility deteriorated paravertebral muscle” and “insufficient lumbar spine flexion”. Therefore, it is possible to achieve a presentation that gives suggestion to the therapist. For example, clinical inference such as those empirically performed by experiential PTs can be reproduced.

The observation reception unit 12 may receive, as an observation, a hypothesis generated from at least one of the hypothesis generation unit 13 and the hypothesis link generation unit 14. For example, by making a hypothesis by assuming the graph structure generated by the hypothesis link generation unit 14 as a new observation, it is possible to generate a graph with higher accuracy.

The above-described motor function improvement assistance apparatus 10 may be, for example, an information processing apparatus such as a personal computer, a server, a portable terminal, or a tablet. The motor function improvement assistance apparatus 10 may include a processor, a memory, a storage device, and a communication device as a configuration (not illustrated). The storage device may store, as a program, processing performed by each component of the motor function improvement assistance apparatus 10. Further, the processor may read the program from the storage device into the memory and execute the program. Thus, the processor implements the functions of each component in the motor function improvement assistance apparatus 10 such as the background knowledge storage unit 11, the observation reception unit 12, the hypothesis generation unit 13, and the hypothesis link generation unit 14. The communication apparatus performs communication necessary for the motor function improvement assistance apparatus 10 to perform information processing.

Each component of the motor function improvement assistance apparatus 10 may be achieved by dedicated hardware. In addition, some or all of the constituent elements may be achieved by a general-purpose or dedicated circuitry, a processor, or the like, or a combination thereof. Some or all of the constituent elements may be constituted by a single chip, or may be constituted by a plurality of chips connected via a bus. Some or all of the constituent elements of each device may be achieved by a combination of the above-described circuit or the like and a program. Further, as the processor, a central processing unit (CPU), a graphics processing unit (GPU), a field-programmable gate array (FPGA), a quantum processor (quantum computer controlled chip), and the like can be used.

In addition, when some or all of the constituent elements of the motor function improvement assistance apparatus 10 are achieved by a plurality of information processing apparatuses, circuits, or the like, the plurality of information processing apparatuses, circuits, or the like may be centrally arranged or arranged in a distributed manner. For example, the information processing apparatus, the circuit, and the like may be achieved by a client server system, a cloud computing system, or the like in a form in which each of the information processing apparatuses, the circuits, and the like are connected via a communication network. In addition, the function of the motor function improvement assistance apparatus 10 may be provided in a software as a service (SaaS) format.

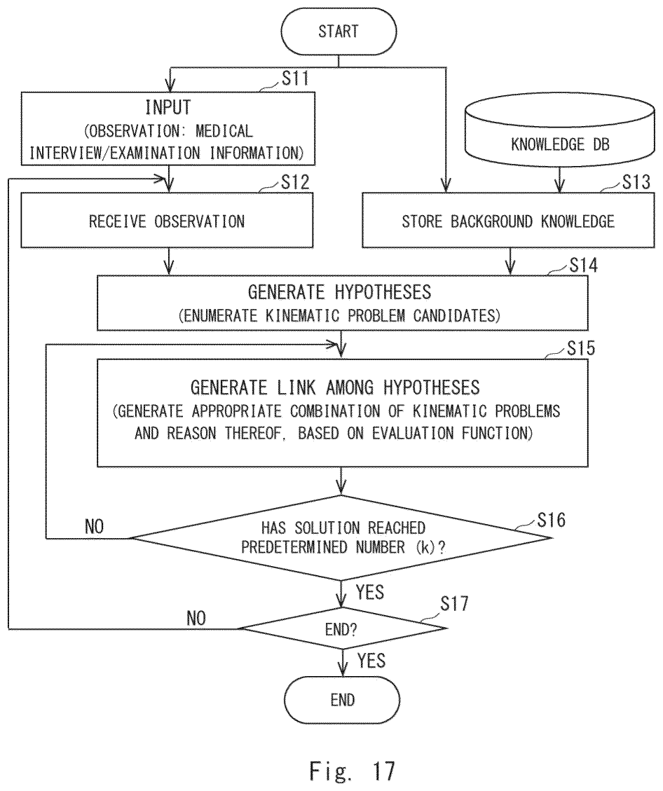

Next, a motor function improvement assistance method is explained. FIG. 17 is a flowchart exemplifying the motor function improvement assistance method according to the first example embodiment. As illustrated in step S11 of FIG. 17, for example, medical interview information and examination information are input as an observation. Next, as illustrated in step S12, an observation including examination information and medical interview information of a subject is received. Specifically, the observation reception unit 12 converts the input observation into a predetermined format that can be read by the information processing apparatus and receives the converted observation.

Meanwhile, as illustrated in step S13, background knowledge is stored. In the background knowledge, the state of the observable body part is associated with a kinematic problem of the state of the observable body part, based on a causal relationship. For example, the background knowledge storage unit 11 converts the background knowledge stored in the knowledge DB into a predetermined format that can be read by the information processing apparatus and stores the converted background knowledge. Steps S11 and S12 and step S13 may be performed in parallel, or one may be performed and the other may be performed thereafter.

Next, as illustrated in step S14, a hypothesis is generated. Specifically, the hypothesis generation unit 13 generates hypotheses of a plurality of kinematic problems by using hypothesis inference, based on background knowledge and observation. The hypothesis generation unit 13 may perform hypothesis inference by combining background knowledge and observation to enumerate a plurality of kinematic problems.

Next, as illustrated in step S15, a hypothesis link is generated. Specifically, the hypothesis link generation unit 14 generates a combination of hypotheses including an interaction between the hypotheses. The hypothesis link generation unit 14 may generate an appropriate combination of kinematic problems and a reason thereof, based on the evaluation function. In steps S14 and S15, by using hypothesis inference of a kind, represented by weighted abduction, that can derive multiple solutions, based on the evaluation function while taking the constraint in view, the hypothesis generation in step S14 and the hypothesis link generation in step S15 can be performed as a set.

Next, as illustrated in step S16, it is determined whether the solution has reached a predetermined number (k). In the case of NO, where the solution has not reached a predetermined number (k), steps S15 and S16 are repeated. Meanwhile, in the case of YES, where the solution has reached a predetermined number (k), it is determined whether processing is to be ended as illustrated in step S17. In step S17, in the case of NO in which the processing is not ended, the observation reception unit 12 may receive the hypothesis generated as a solution as an observation. Then, steps S12 to S17 may be repeated. Meanwhile, in step S17, in the case of YES in which the processing is to be ended, the processing is ended.

Next, effects of the present example embodiment is described. The motor function improvement assistance apparatus 10 of the present example embodiment can reproduce clinical inference routinely performed by a PT by outputting constrained interaction of kinematic problems as a graph structure from the medical interview/examination information. Therefore, the motor function of the subject can be effectively improved.

In addition, by visualizing the reproduced clinical inference as a graph structure, an overall picture of a physical condition of a patient who has a chief complaint of lower back pain or the like is revealed to anyone's eyes, and it becomes possible to easily understand which movement of which part should be improved.

Since each of the interactions among kinematic problems is visualized, the ripple effect of the intervention on each kinematic problem becomes also visible. Therefore, it becomes possible to assist establishment of a motor function improvement plan including rehabilitation.

Consistent interventions can be performed since an overview of the patient's kinematic problems is presented independently of the experience and competence of the PT.

So far, it has been desired to solve the problem of clarifying constrained interactions between a plurality of kinematic problems from limited medical interview information and examination information to assist consistent motor function improvement. However, the motor function improvement assistance apparatus 10 according to the present example embodiment can clarify the constrained interaction between a plurality of kinematic problems from limited medical interview information and examination information by applying a hypothesis inference technique. Meanwhile, for example, a related automatic diagnosis technique presents a highly probable pathological condition/disease name from a given medical interview information, which can be solved by using a general AI technique to solve a discrimination problem or a classification problem. However, in the automatic diagnosis field, there is no example in which a hypothesis inference technique is applied, and there is no problem setting as described above. The present example embodiment can solve the above-described problem by applying the hypothesis inference technique for assisting motor function improvement.

### Second Example Embodiment

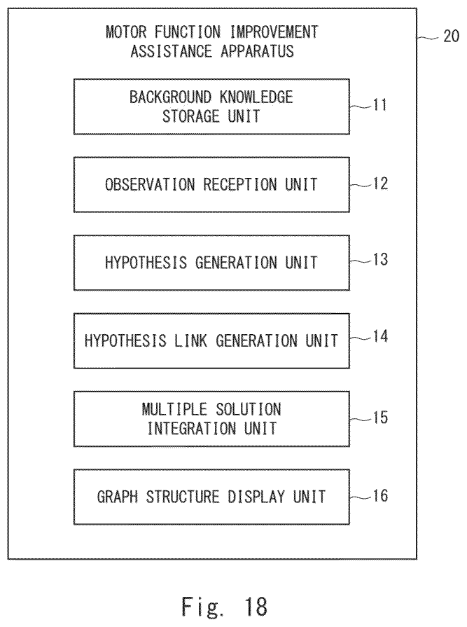

Next, a second example embodiment is explained. FIG. 18 is a block diagram exemplifying a motor function improvement assistance apparatus according to the second example embodiment. As illustrated in FIG. 18, a motor function improvement assistance apparatus 20 according to the present example embodiment further includes a multiple solution integration unit 15 and a graph structure display unit 16 as compared with the motor function improvement assistance apparatus 10 described above. The multiple solution integration unit 15 and the graph structure display unit 16 have functions as a multiple solution integration means and a graph structure display means.

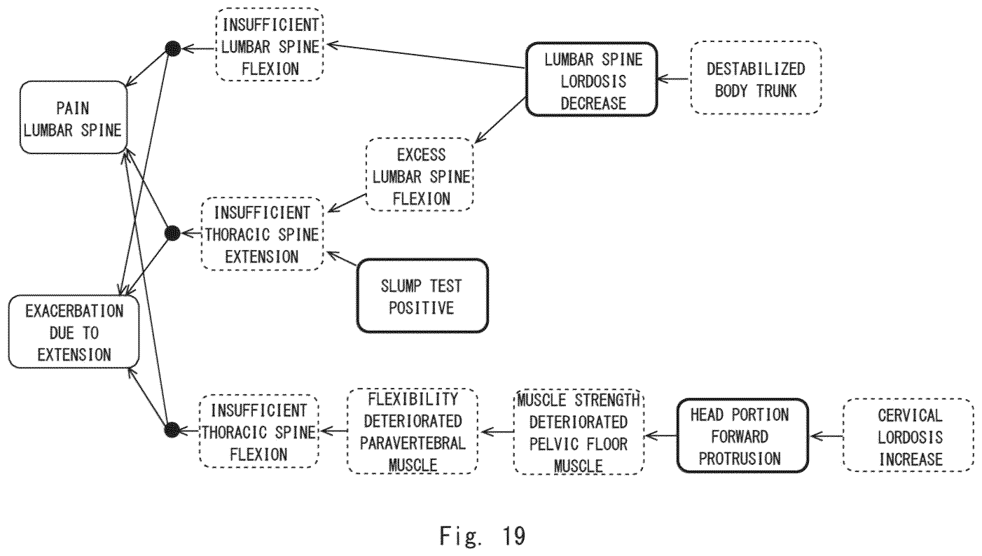

The multiple solution integration unit 15 integrates the generated plurality of hypotheses. Specifically, the multiple solution integration unit 15 integrates the acquired multiple solutions. FIG. 19 is a diagram exemplifying a plurality of integrated hypotheses in the motor function improvement assistance apparatus 20 according to the second example embodiment. As illustrated in FIG. 19, for example, the multiple solution integration unit 15 visualizes a plurality of hypotheses having the same evaluation function value of the inference result together as single set. For example, the multiple solution integration unit 15 may visualize integrated hypotheses of FIGS. 14 to 16.

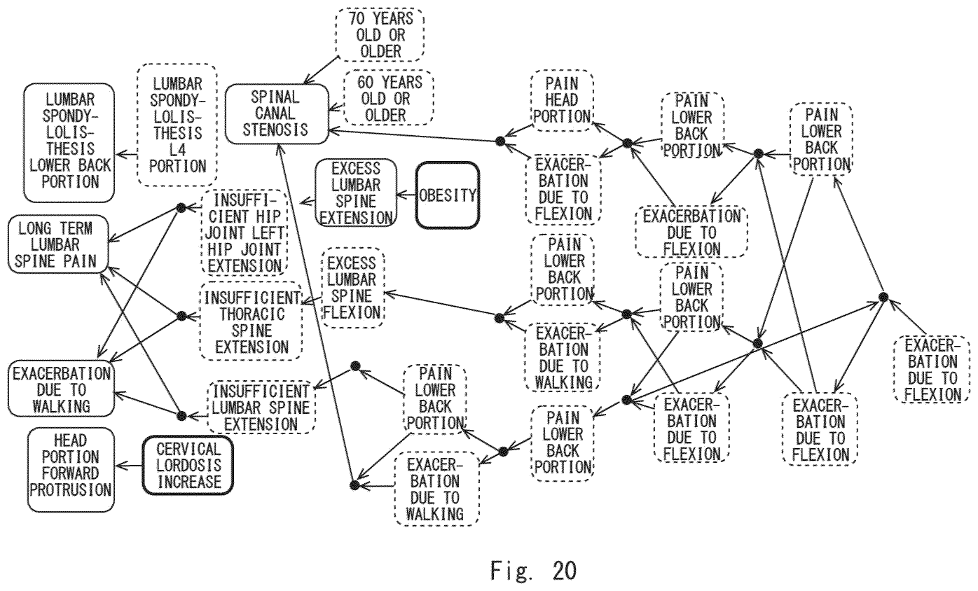

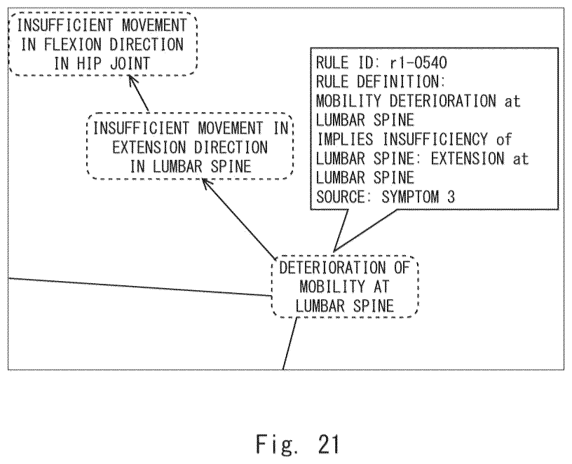

FIGS. 20 and 21 are diagrams exemplifying visualized integrated graph structures in the motor function improvement assistance apparatus 20 according to the second example embodiment. As illustrated in FIG. 20, the graph structure display unit 16 generates an integrated hypothesis as a graph structure. Then, the graph structure display unit 16 visualizes and displays the graph structure. For example, kinematic problems hypothesized from medical interview information and examination information (items surrounded by thin lines and thick lines) and relationships therebetween are visualized as a graph. When the acquired hypothesis is incorrect or a new test result for the state suggested by the hypothesis is acquired, correction or new items can be added to the observation and inferred again as described in Patent Literature 5. As illustrated in FIG. 21, in the graph structure displayed by the graph structure display unit 16, additional information such as source information of background knowledge may be displayed by, for example, clicking on an edge or a node.

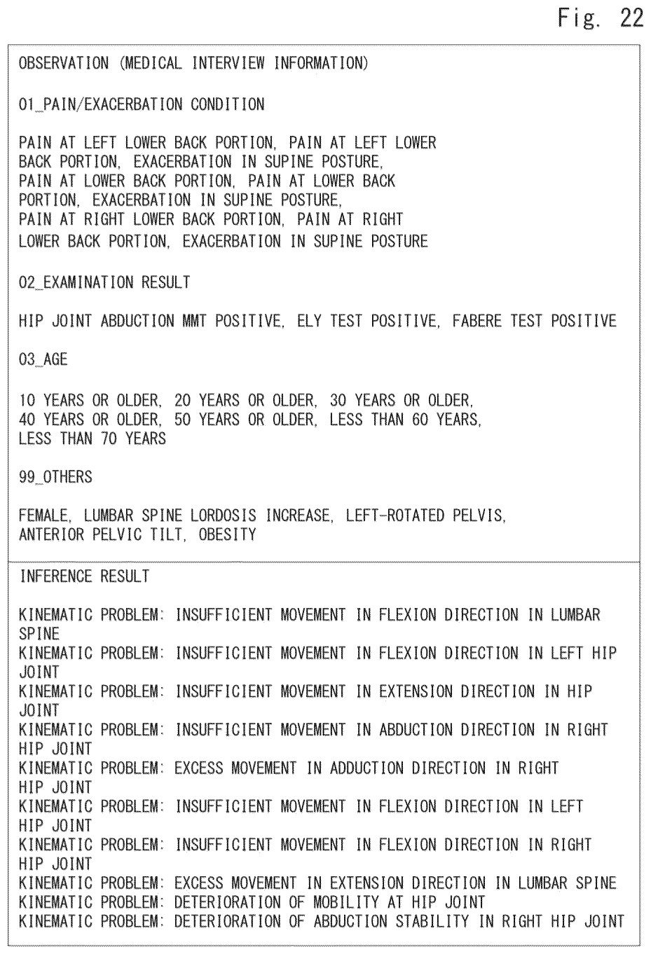

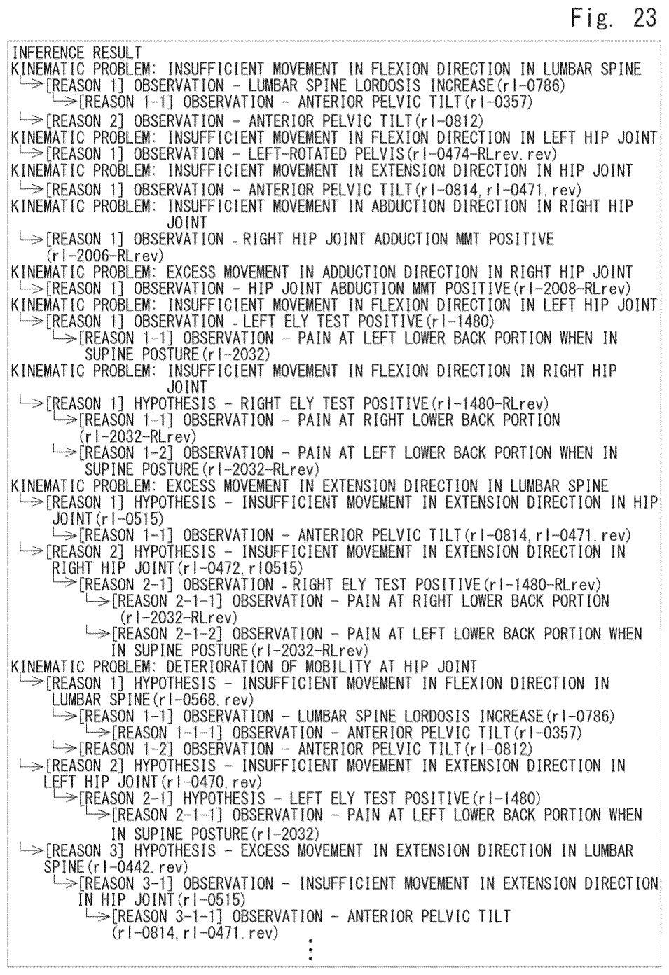

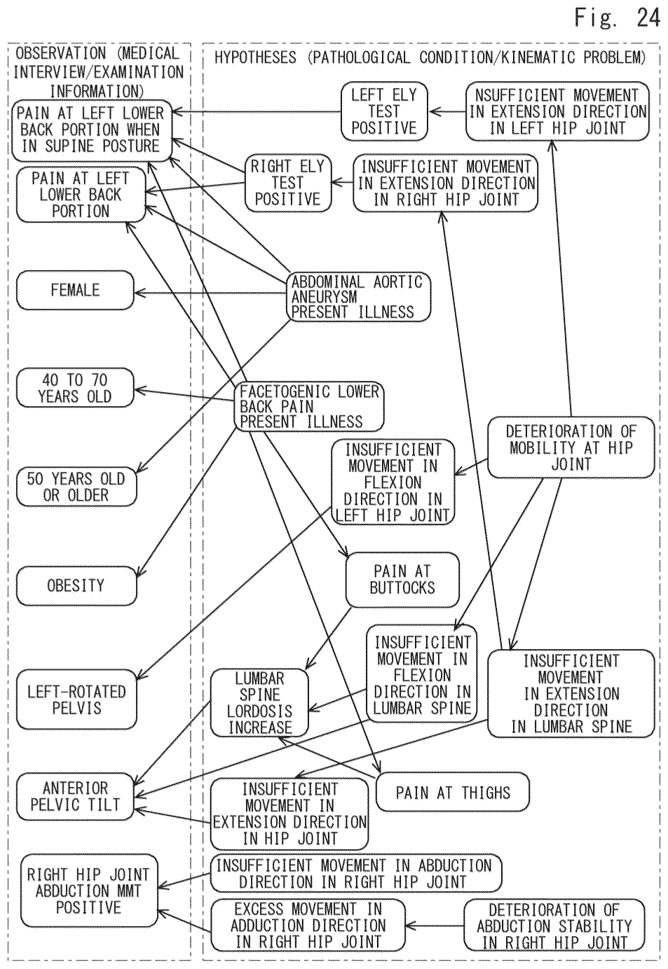

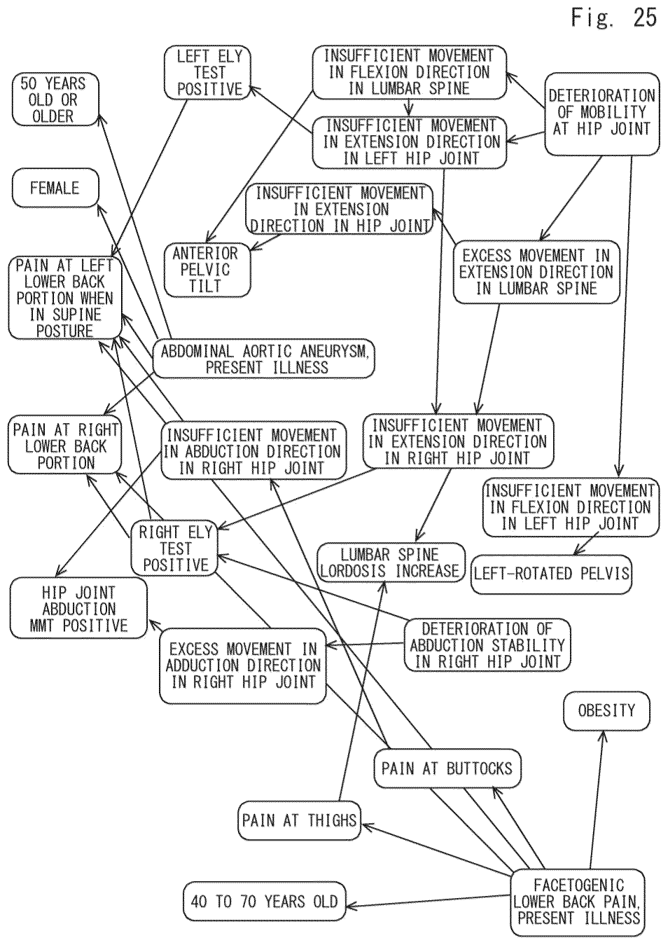

Also, the links between the integrated hypotheses are not limited to the graphical representation by the graph structure. FIGS. 22 and 23 are diagrams exemplifying visualized inference results in the motor function improvement assistance apparatus according to the second example embodiment. FIGS. 24 and 25 are diagrams exemplifying visualized graph structures in the motor function improvement assistance apparatus according to the second example embodiment. As illustrated in FIGS. 22 and 23, the links between the integrated hypotheses may be displayed as a report by using a means such as natural sentences or bullets as well as a graph structure. For example, a desired hypothesis (in this case, a kinematic problem) and underlying observations and hypotheses may be displayed using natural sentences or bullets. Note that, as illustrated in FIGS. 24 and 25, a display method of the graph structure, for example, separately visualizing an observation (medical interview/examination information) and a hypothesis (pathological condition/kinematic problem), may be selected as appropriate.

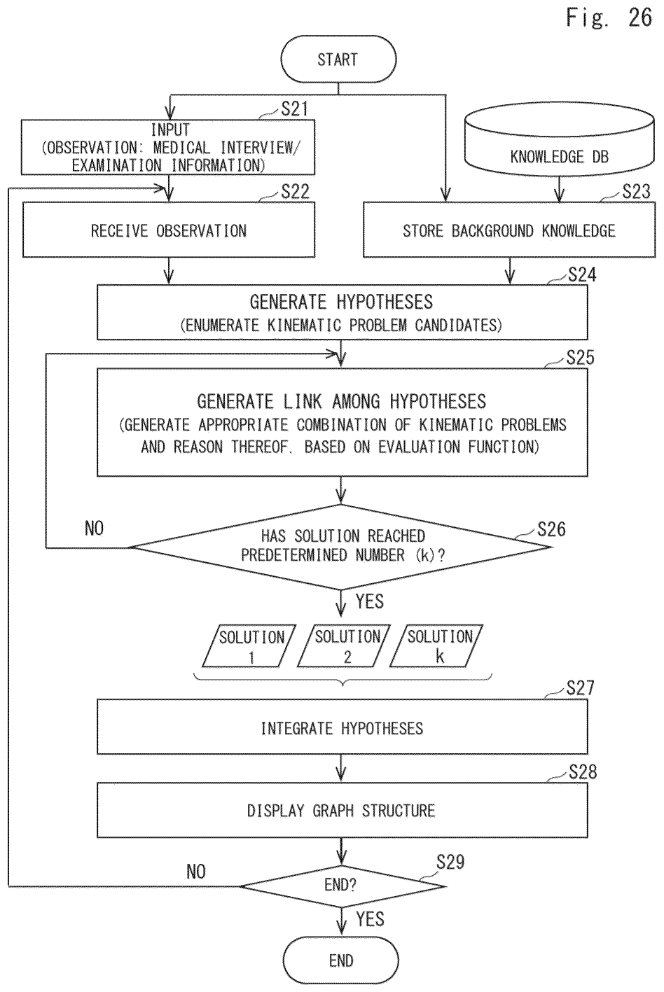

Next, a motor function improvement assistance method according to the second example embodiment is explained. FIG. 26 is a flowchart exemplifying a motor function improvement assistance method according to the second example embodiment. Steps S21 to S26 in FIG. 26 are similar to steps S11 to S16 in FIG. 17.

In step S26, in the case of NO, where the solution has not reached a predetermined number (k), steps S25 and S26 are repeated. In the case of YES, where the solution has reached the predetermined number (k), a plurality of hypotheses are integrated as illustrated in step S27. Specifically, the multiple solution integration unit 15 integrates the generated plurality of hypotheses. Next, as illustrated in step S28, a graph structure is displayed. Specifically, the graph structure display unit 16 generates a graph structure of the integrated hypothesis.

Next, as illustrated in step S29, it is determined whether the processing is to be ended. In step S29, in the case of NO in which the processing is not to be ended, the observation reception unit 12 may receive the hypothesis generated as a solution as an observation. Then, steps S22 to S29 may be repeated. Meanwhile, in step S29, in the case of YES in which the processing is to be ended, the processing is ended.

Next, effects of the present example embodiment is explained. According to the present example embodiment, interactions among each kinematic problem can be visualized as a graph structure. Thus, the patient's overall kinematic problems can be presented more clearly regardless of the experience and competence of the PT, thus allowing for a consistent intervention. Other configurations and effects are included in the description of the first example embodiment.

The present disclosure is not limited to the above-described example embodiments, and can be appropriately modified without departing from the sprit and scope of the present disclosure. For example, the configurations of the first and second example embodiments may be combined.

In addition, a motor function improvement assistance program for causing a computer to read and execute the above-described motor function improvement assistance method is also within the scope of the technical idea of the example embodiments. The motor function improvement assistance program may be stored in a non-transitory computer-readable medium or a tangible storage medium. By way of example, and not limitation, computer-readable media or tangible storage media include a random-access memory (RAM), a read-only memory (ROM), a flash memory, a solid-state drive (SSD) or other memory technologies, a CD-ROM, a digital versatile disc (DVD), a Blu-ray (registered trademark) disk, or other optical disk storages, a magnetic cassette, a magnetic tape, a magnetic disk storage or other magnetic storage devices. The information processing program may be transmitted via a transitory computer readable medium or via a communication medium. By way of example, and not limitation, the transitory computer-readable media or communication media include electrical, optical, acoustic, or other forms of propagated signals.

Some or all of the above-described embodiments may be described as the following supplementary notes, but are not limited thereto.

### Supplementary note A1

A motor function improvement assistance method including:
storing background knowledge in which a state of an observable body part and a kinematic problem of the state are associated with each other based on a causal relationship;
receiving an observation including examination information and medical interview information of a subject;
generating, based on the background knowledge and the observation, hypotheses of a plurality of kinematic problems by using hypothesis inference; and
generating a combination of hypotheses including an interaction between the hypotheses.

### Supplementary note A2

The motor function improvement assistance method according to supplementary note A1, further including:
integrating the generated hypotheses; and
generating a graph structure from the integrated hypotheses.

### Supplementary note A3

The motor function improvement assistance method according to supplementary note A1 or A2, further including, when receiving the observation, receiving the generated hypothesis as the observation.

### Supplementary note A4

The motor function improvement assistance method according to any one of supplementary notes A1 to A3, further including:
when storing the background knowledge, storing an exclusive relationship among a plurality of the kinematic problems that are not simultaneously established, as the background knowledge; and
when generating the hypothesis, generating the hypotheses, based on the background knowledge including the exclusive relationship.

### Supplementary note A5

The motor function improvement assistance method according to any one of supplementary notes A1 to A4, further including, when generating the hypothesis, evaluating each of the generated hypotheses by using an evaluation function for evaluating the hypothesis.

### Supplementary note A6

The motor function improvement assistance method according to any one of supplementary notes A1 to A5, further including, when generating the hypothesis, generating the hypothesis by weighted abduction applying a backward reasoning operation and a unification operation.

### Supplementary note A7

The motor function improvement assistance method according to any one of supplementary notes A1 to A6, further including formulating as an integer linear programming problem or a satisfiability problem when generating the hypothesis, thereby generating the hypothesis of the plurality of kinematic problems.

### Supplementary note A8

The motor function improvement assistance method according to any one of supplementary notes A1 to A7, further including, when generating the hypothesis, using a solver capable of outputting multiple solutions and thus generating a plurality of the hypotheses having the same evaluation function value for evaluating the generated plurality of hypotheses.

### Supplementary note B1

A motor function improvement assistance program that causes a computer to execute:
storing background knowledge in which a state of an observable body part and a kinematic problem of the state are associated with each other based on a causal relationship;
receiving an observation including examination information and medical interview information of a subject;
generating, based on the background knowledge and the observation, hypotheses of a plurality of kinematic problems by using hypothesis inference; and
generating a combination of the hypotheses including an interaction between the hypotheses.

### Supplementary note B2

The motor function improvement assistance program according to supplementary note B1 that causes the computer to further execute:
integrating the generated hypotheses; and
generating a graph structure from the integrated hypotheses.

### Supplementary note B3

The motor function improvement assistance program according to supplementary note B1 or B2 that causes the computer to execute, when receiving the observation, receiving the generated hypothesis as the observation.

### Supplementary note B4

The motor function improvement assistance program according to any one of supplementary notes Bi to B3 that causes the computer to execute:
when storing the background knowledge, storing, as the background knowledge, an exclusive relationship among a plurality of the kinematic problems that are not simultaneously established; and
when generating the hypothesis, generating the hypotheses, based on the background knowledge including the exclusive relationship.

### Supplementary note B5

The motor function improvement assistance program according to any one of supplementary notes B1 to B4 that causes the computer to execute, when generating the hypothesis, evaluating each of the generated hypotheses by using an evaluation function for evaluating the hypothesis.

### Supplementary note B6

The motor function improvement assistance program according to any one of supplementary notes B1 to B5 that causes the computer to execute, when generating the hypothesis, generating the hypothesis by weighted abduction applying a backward reasoning operation and a unification operation.

### Supplementary note B7

The motor function improvement assistance program according to any one of supplementary notes B1 to B6 that causes the computer to execute, when generating the hypothesis, formulating as an integer linear programming problem or a satisfiability problem and thereby generating the hypothesis of the plurality of kinematic problems.

### Supplementary note B8

The motor function improvement assistance program according to any one of supplementary notes B1 to B7 that causes the computer to execute, when generating the hypothesis, using a solver capable of outputting multiple solutions and thus generating a plurality of the hypotheses having the same evaluation function value for evaluating the generated plurality of hypotheses.

Although the present invention has been described with reference to the example embodiments, the present invention is not limited to the above. Various modifications that can be understood by a person skilled in the art within the scope of the invention can be made to the configuration and details of the present invention.

This application claims priority based on Japanese Patent Application No. 2022-058197, filed on Mar. 31, 2022, the disclosure of which is incorporated herein in its entirety.

### REFERENCE SIGNS LIST

10, 20 MOTOR FUNCTION IMPROVEMENT ASSISTANCE APPARATUS
11 BACKGROUND KNOWLEDGE STORAGE UNIT
12 OBSERVATION RECEPTION UNIT
13 HYPOTHESIS GENERATION UNIT
14 HYPOTHESIS LINK GENERATION UNIT
15 MULTIPLE SOLUTION INTEGRATION UNIT
16 GRAPH STRUCTURE DISPLAY UNIT

1. A motor function improvement assistance apparatus comprising:
background knowledge storage unit configured to store background knowledge in which a state of an observable body part and a kinematic problem of the state are associated with each other based on a causal relationship;
observation reception unit configured to receive an observation including examination information and medical interview information of a subject;
hypothesis generation unit configured to generate, based on the background knowledge and the observation, hypotheses of a plurality of kinematic problems by using hypothesis inference; and
hypothesis link generation unit configured to generate a combination of hypotheses including an interaction between the hypotheses.

2. The motor function improvement assistance apparatus according to claim 1, further comprising:
multiple solution integration unit configured to integrate the generated hypotheses; and
graph structure display unit configured to generate a graph structure from the integrated hypotheses.

3. The motor function improvement assistance apparatus according to claim 1, wherein the observation reception unit receives the generated hypotheses as the observation.

4. The motor function improvement assistance apparatus according to claim 1, wherein
the background knowledge storage unit stores, as the background knowledge, an exclusive relationship among a plurality of the kinematic problems that are not established simultaneously, and
the hypothesis generation unit generates the hypotheses, based on the background knowledge including the exclusive relationship.

5. The motor function improvement assistance apparatus according to claim 1, wherein the hypothesis generation unit evaluates each of the generated hypotheses by using an evaluation function for evaluating the hypothesis.

6. The motor function improvement assistance apparatus according to claim 1, wherein the hypothesis generation unit generates the hypothesis by weighted abduction applying a backward inference operation and a unification operation.

7. The motor function improvement assistance apparatus according to claim 1, wherein the hypothesis generation unit generates the hypothesis of a plurality of the kinematic problems by using formulation into an integer linear programming problem or a satisfiability problem.

8. The motor function improvement assistance apparatus according to claim 1, wherein the hypothesis generation unit uses a solver capable of outputting multiple solutions and generates a plurality of the hypotheses having the same evaluation function value for evaluating the generated plurality of hypotheses.

9. A motor function improvement assistance method comprising:
storing background knowledge in which a state of an observable body part and a kinematic problem of the state are associated with each other based on a causal relationship;
receiving an observation including examination information and medical interview information of a subject;
generating, based on the background knowledge and the observation, hypotheses of a plurality of kinematic problems by using hypothesis inference; and
generating a combination of hypotheses including an interaction between the hypotheses.

10. The motor function improvement assistance method according to claim 9, further comprising:
integrating the generated hypotheses; and
generating a graph structure from the integrated hypotheses.

11. The motor function improvement assistance method according to claim 9, further comprising, when receiving the observation, receiving the generated hypothesis as the observation.

12. The motor function improvement assistance method according to claim 9, further comprising:
when storing the background knowledge, storing an exclusive relationship among a plurality of the kinematic problems that are not simultaneously established, as the background knowledge; and,
when generating the hypothesis, generating the hypothesis, based on the background knowledge including the exclusive relationship.

13. The motor function improvement assistance method according to claim 9, further comprising, when generating the hypothesis, evaluating each of the generated hypotheses by using an evaluation function for evaluating the hypothesis.

14. The motor function improvement assistance method according to claim 9, further comprising, when generating the hypothesis, generating the hypothesis by weighted abduction applying a backward reasoning operation and a unification operation.

15. The motor function improvement assistance method according to claim 9, further comprising formulating as an integer linear programming problem or a satisfiability problem when generating the hypothesis, thereby generating the hypothesis of the plurality of kinematic problems.

16. The motor function improvement assistance method according to claim 9, further comprising, when generating the hypothesis, using a solver capable of outputting multiple solutions thereby generating a plurality of the hypotheses having the same evaluation function value for evaluating the generated plurality of hypotheses.

17. A non-transitory computer-readable medium storing a motor function improvement assistance program that causes a computer to execute:
storing background knowledge in which a state of an observable body part and a kinematic problem of the state are associated with each other based on a causal relationship;
receiving an observation including examination information and medical interview information of a subject;
generating, based on the background knowledge and the observation, hypotheses of a plurality of kinematic problems by using hypothesis inference; and
generating a combination of hypotheses including an interaction between the hypotheses.

18. The non-transitory computer-readable medium storing the motor function improvement assistance program according to claim 17 that causes the computer to further execute:
integrating the generated hypotheses; and
generating a graph structure from the integrated hypotheses.

19. The non-transitory computer-readable medium storing the motor function improvement assistance program according to claim 17 that causes the computer to execute, when receiving the observation, receiving the generated hypothesis as the observation.

20. The non-transitory computer-readable medium storing the motor function improvement assistance program according to claim 17 that causes the computer to execute:
when storing background knowledge, storing, as the background knowledge, an exclusive relationship among a plurality of the kinematic problems that are not simultaneously established; and
when generating the hypothesis, generating the hypothesis, based on the background knowledge including the exclusive relationship.

21-24. (canceled)
